(19) United States
(12) Patent Application Publication
(10) Pub. No.: US 2009/0138092 A1
(43) Pub. Date: May 28, 2009

(54) Therapeutic Structures for Utilization in Temporomandibular Joint Replacement Systems

(76) Inventors: Brent W. Johnston, Spokane, WA (US); George Madison Sims, IV, Shavano Park, TX (US)

Correspondence Address: WELLS ST. JOHN P.S., 601 W. FIRST AVENUE, SUITE 1300, SPOKANE, WA, 99201, US

(21) Appl. No.: 12/277,200

(22) Filed: Nov. 24, 2008

Related U.S. Application Data
(60) Provisional application No. 60/990,789, filed on Nov. 28, 2007.

(51) Int. Cl. A61F 2/02; A61B 17/70; A61B 17/04

(52) U.S. Cl. 623/17.17; 606/280; 606/286; 606/301

(57) ABSTRACT

The invention includes constructions suitable for utilization in temporomandibular joint replacement systems, with the constructions being formed of pyrocarbon-coated material. The pyrocarbon-coated material may be, for example, a pyrocarbon-coated tungsten/graphite matrix.

### RELATED PATENT DATA

This application is related to U.S. Provisional Application Ser. No. 60/990,789; which was filed Nov. 28, 2007.

### TECHNICAL FIELD

The invention pertains to therapeutic structures for utilization in temporomandibular joint replacement systems; and to methods of making and using therapeutic structures in temporomandibular joint replacement systems.

### BACKGROUND

The human mandible is a U-shaped bone (lower jaw bone) having a generally horizontal body portion with an anterior prominence (mandibular symphysis) defining the chin in the facial structure. A posterior branch or ramus extends upwardly and rearwardly from each end of the body portion. The upper end of each ramus terminates in a forward coronoid process and a rearward condyloid process or condyle. The condyle is a knob-shaped prominence which fits into a cup-shaped socket known as the glencid fossa formed in the temporal bone of the skull. The condyle, glenoid fossa, and supporting muscle tissue on each side of the skull define a temporomandibular joint (TMJ) which permits the lower jaw to be freely movable.

Various portions of the TMJ may be damaged through disease and/or injury, and accordingly therapeutic structures have been developed for replacing part or all of the skeletal structure of a TMJ. Conventional therapeutic structures utilized in temporomandibular joint replacement systems are expensive to manufacture and/or lack desired endurance. Accordingly, it is desired to develop new therapeutic structures suitable for utilization in temporomandibular joint replacement systems. It is desired for the new therapeutic structures to have high biocompatibility, high strength, low weight, and good durability.

### SUMMARY OF THE INVENTION

In one aspect, the invention includes a therapeutic structure comprising pyrocarbon-coated material, and configured for utilization in a temporomandibular joint replacement system. The material may be, for example, a tungsten/graphite matrix. The structure may be for example, a fossa prosthesis or a mandibular prosthesis.

### BRIEF DESCRIPTION OF THE DRAWINGS

FIG. 1 is a diagrammatic view of example embodiment therapeutic structures utilized in a temporomandibular joint replacement system.

FIG. 2 is a diagrammatic view of an example embodiment fossa prosthesis.

FIG. 3 is a diagrammatic view of an example embodiment mandibular prosthesis.

FIG. 4 is a diagrammatic view of an example embodiment mandibular prosthesis movably joined with an example embodiment fossa prosthesis.

### DETAILED DESCRIPTION OF THE ILLUSTRATED EMBODIMENTS

The temporomandibular joint system comprises a combination of bones, muscles, cartilage, nerves and fluids that work together to permit movement of the jaw. If structural damage to the temporomandibular joint system occurs, then various therapeutic structures may be attached to the jaw and/or other bones to provide support within the temporomandibular joint system. The invention includes therapeutic structures that may be utilized in temporomandibular joint replacement systems. The structures may include plates and/or fasteners (with example fasteners being screws). The fasteners can contain pores therein, with the pores being configured so that bone structure grows into the pores to improve union of the fasteners with bone. The bone structure growth into the pores can be enhanced by providing one or more bone-growth-stimulating compositions within the pores. Additionally, or alternatively, bone cement can be provided within the pores. Example screws having pores therein are described in U.S. Patent Publication No. 2007/0154514.

The various therapeutic structures of the present invention can comprise carbon/metal matrices (in other words, can comprise matrices which include both carbon and metal). The carbon/metal matrices can comprise any suitable composition or combination of compositions. In some aspects, the carbon of the carbon/metal matrices can be in the form of graphite, and the metal can comprise a transition metal, such as, for example, a group 6 (new IUPAC notation) metal, such as tungsten. In example aspects, the metal of a carbon/metal matrix of the invention can comprise, consist essentially of, or consist of tungsten. For instance, the carbon/metal matrices can consist essentially of, or consist of graphite/tungsten (with “graphite/tungsten” being understood to mean graphite and tungsten); with the tungsten being present to from about 1 weight % to about 30 weight %; typically from about 5 weight % to about 20 weight %; and most typically to about 10 weight %.

The carbon/metal matrices can be at least partially encapsulated with pyrocarbon (in other words, can be at least partially coated with a pyrolytic coating), or can be otherwise treated to form pyrocarbon that extends within the matrices and/or across surfaces of the matrices. The carbon/metal matrices may be substantially entirely encapsulated, or even entirely encapsulated with pyrocarbon to enhance biocompatibility of the structures. The pyrocarbon can be provided to a thickness of at least about 0.01 inch; and can be formed as described in U.S. Pat. Nos. 5,514,410; 5,641,324; 6,217,616 and 5,843,183; and/or as available as On-X™ carbon from Medical Research Carbon Institute (MCRI™) of Austin, Tex. U.S.A.

In some aspects, therapeutic structures described herein can be formed of any suitable pyrocarbon-coated material. The material can be, for example, a metal-containing material, such as a carbon/metal matrix of the type described above.

In some aspects, the present invention includes a recognition that the biocompatibility and strength-to-weight properties of pyrocarbon-coated metal/carbon matrices (for instance, the carbon/tungsten matrices discussed above) can be of particular advantage for utilization in structures comprised by temporomandibular joint replacement systems.

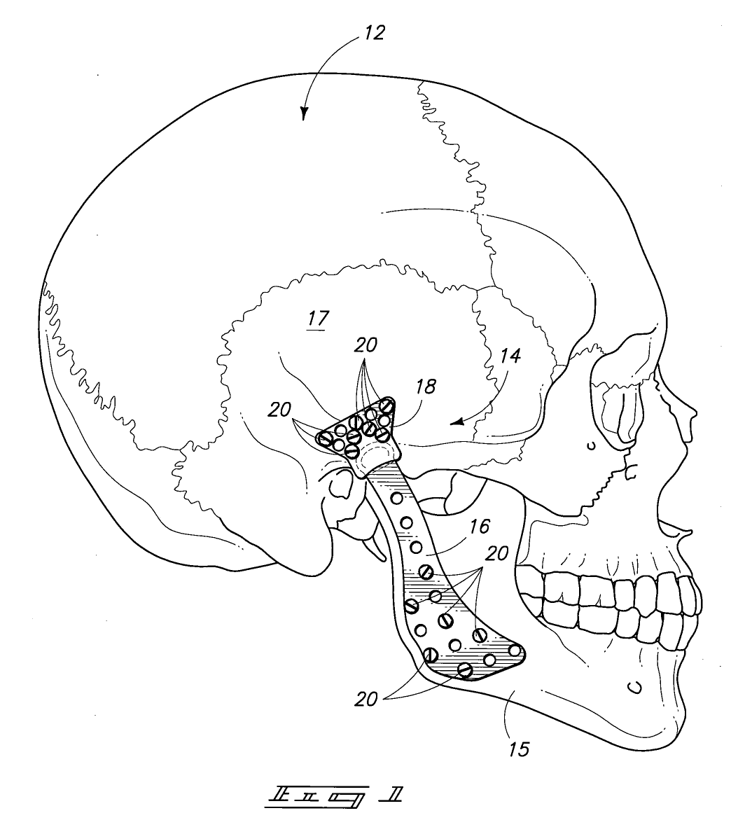

Example aspects of the invention are described below with reference to FIGS. 1-4. FIG. 1 illustrates a skull 12 having a temporomandibular joint replacement system 14 attached thereto. The temporomandibular joint replacement system 14 comprises a mandibular (or condylar) prosthesis 16 and a fossa prosthesis 18. Either or both of the mandibular prosthesis 16 and fossa prosthesis 18 may be formed of a pyrocarbon-coated metal/carbon matrix. The mandibular prosthesis is attached to a mandible (or jawbone) 15, and the fossa prosthesis is attached to a temporal bone 17.

It can be advantageous for one or both of mandibular prosthesis 16 and fossa prosthesis 18 to comprise a carbon/metal matrix coated with pyrocarbon. The pyrocarbon can form a biocompatible coating. Additionally, the processing to form the pyrocarbon can significantly alter characteristics of the carbon/metal matrix to create much more strength within the carbon/metal matrix than would be present without such processing. Although the pyrocarbon is referred to as a coating, it is to be understood that the processing utilized to form the pyrocarbon can create changes throughout the carbon/metal matrix, as well as at the surface.

The mandibular prosthesis 16 and fossa prosthesis 18 are retained to the skull with a plurality of fasteners 20. The fasteners may be any suitable fasteners, and in the shown application are screws. The fasteners may be conventional fasteners, or may comprise pyrocarbon-coated material; and/or comprise a carbon/metal matrix. The fasteners may, for example, comprise, consist essentially of, or consist of a carbon/metal matrix, either alone, or coated with pyrocarbon. It can be advantageous to utilize a carbon/metal matrix coated with pyrocarbon for the reasons discussed above. In some embodiments, the fasteners may be screws having pores extending therein suitable to enable bone growth to penetrate the screws and assist in retaining the screws to bone structures.

The fasteners utilized to retain the fossa prosthesis to the temporal bone may be referred to as first set of fasteners, and the fasteners utilized to retain mandibular prosthesis to the jawbone may be referred to as a second set of fasteners. The fasteners of the first set may be the same as those of the second set, or may be different from those of the second set.

FIG. 2 shows the fossa prosthesis 18 of FIG. 1 in isolation from the remainder of the temporomandibular joint replacement system. The fossa prosthesis comprises a plurality of openings 20 extending therethrough. In operation, fasteners (such as screws, rivets, etc.) may be passed through some or all of the openings to retain the fossa prosthesis to the temporal bone of a human skull.

The fossa prosthesis also comprises a receptacle, or cup region, 22 configured to receive a projection from a mandibular prosthesis.

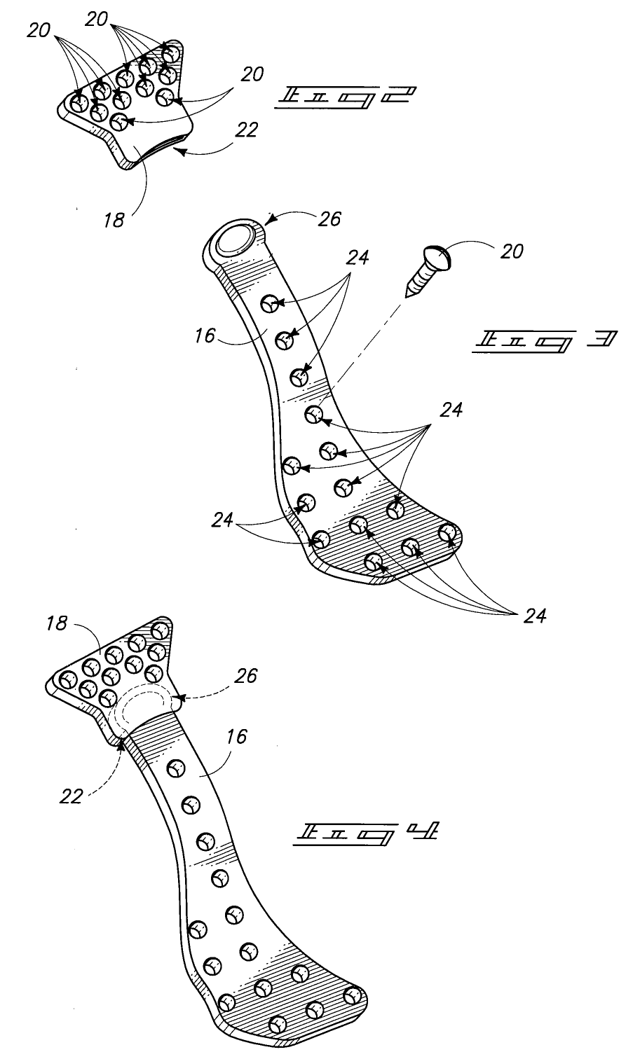

FIG. 3 shows the mandibular prosthesis 16 of FIG. 1 in isolation from the remainder of the temporomandibular joint replacement system. The mandibular prosthesis comprises a plurality of openings 24 extending therethrough. In operation, fasteners (such as screws, bolts, rivets, etc.) may be passed through some or all of the openings to retain the mandibular prosthesis to the jawbone of a human skull.

The mandibular prosthesis comprises a protruding region 26 that forms a projection configured to extend into the cup region 22 of the fossa projection to form a movable joint. The protruding region may be considered to substitute for the mandibular condyle in some embodiments.

FIG. 4 shows the mandibular prosthesis 16 movably joined to the fossa prostheses 18 through engagement of the protruding region 26 of the mandibular prosthesis within the cup region 22 of the fossa prosthesis.

It may be advantageous to form the mandibular prosthesis and fossa prosthesis of a TMJ replacement system to be of pyrocarbon-coated metal/carbon matrices, in that pyrocarbon-coated metal/carbon matrices have excellent biocompatibility, strength-to-weight ratio, and wear properties. Accordingly, the TMJ replacement will be comfortable and long lasting.

A problem with conventional TMJ replacement systems is that the joint formed at the interface of the fossa prosthesis and mandibular prosthesis tends to wear out due to the high amount of repetitive wear that the joint is subjected to. The excellent wear properties of pyrocarbon-coated metal/carbon matrices may extend the life of the joint relative to various prior art materials conventionally utilized in TMJ replacement systems, which can be of significant benefit to persons wearing the TMJ replacement systems. Additionally, the high strength-to-weight ratio of pyrocarbon-coated metal/carbon matrices may enable TMJ replacement systems of the present invention to be made more lightweight and thin than conventional systems, which can improve the comfort of TMJ replacement systems of the present invention relative to conventional TMJ replacement systems; which can be of significant benefit to persons wearing the TMJ replacement systems.

The improvements in weight and longevity of TMJ replacement systems of the present invention may be enhanced by utilizing fasteners consisting of pyrocarbon-coated metal/carbon matrices.

In some embodiments, it may be advantageous for the fasteners to have pores extending therein and configured to receive bone growth to assist in retaining the fasteners to a skeletal structure. For instance, the skeletal structure may be thin or otherwise compromised from disease and/or injury, and thus the fasteners may be difficult retain to the skeletal structure over a long term in the absence of bone growth extending into pores in the fasteners. This difficulty may be particularly severe for TMJ replacement structures due to the load placed on the structures when persons wearing the structures chew or otherwise clench their jaw.

In compliance with the statute, the subject matter disclosed herein has been described in language more or less specific as to structural and methodical features. It is to be understood, however, that the claims are not limited to the specific features shown and described, since the means herein disclosed comprise example embodiments. The claims are thus to be afforded full scope as literally worded, and to be appropriately interpreted in accordance with the doctrine of equivalents.

I/we claim:

1. A temporomandibular joint replacement system comprising at least one structure that contains a pyrocarbon-coated matrix.

2. The system of claim 1 wherein the at least one structure includes a mandibular prosthesis.

3. The system of claim 1 wherein the at least one structure includes a fossa prosthesis.

4. The system of claim 1 wherein the matrix comprises carbon and tungsten.

5. The system of claim 4 wherein the matrix consists of carbon and tungsten.

6. The system of claim 5 wherein the matrix comprises from about 1 weight % tungsten to about 30 weight % tungsten.

7. A mandibular prosthesis consisting of a pyrocarbon-coated carbon/metal matrix.

8. The mandibular prosthesis of claim 7 wherein the matrix consists of carbon and tungsten.

9. A fossa prosthesis comprising a pyrocarbon-coated carbon/metal matrix.

10. The fossa prosthesis of claim 9 wherein the matrix consists of carbon and tungsten.

11. A temporomandibular joint replacement system comprising:
a fossa prosthesis having a cup region extending therein;
a mandibular prosthesis having a protruding region that movably engages the cup region of the fossa prosthesis;
a first set of fasteners anchoring the fossa prosthesis to a temporal bone of a human skull;
a second set of fasteners anchoring the mandibular prosthesis to a human jawbone; and
wherein the mandibular prostheses consists of a pyrocarbon-coated metal/carbon matrix.

12. The joint replacement system of claim 11 wherein the metal of the metal/carbon matrix predominately comprises tungsten.

13. The joint replacement system of claim 11 wherein the metal/carbon matrix consists of tungsten and carbon, and comprises from about 1 weight % tungsten to about 30 weight % tungsten.

14. The joint replacement system of claim 11 wherein the fossa prosthesis comprises a pyrocarbon-coated metal/carbon matrix.

15. The joint replacement system of claim 11 wherein individual fasteners of the first set of fasteners comprise a pyrocarbon-coated metal/carbon matrix.

16. The joint replacement system of claim 11 wherein the first set of fasteners are screws formed of a pyrocarbon-coated metal/carbon matrix.

17. The joint replacement system of claim 16 wherein the screws comprise pores extending therein configured for enabling bone growth to extend into the pores to assist in retaining the screws to the temporal bone.

18. The joint replacement system of claim 11 wherein individual fasteners of the second set of fasteners comprise a pyrocarbon-coated metal/carbon matrix.

19. The joint replacement system of claim 11 wherein the second set of fasteners are screws formed of a pyrocarbon-coated metal/carbon matrix.

20. The joint replacement system of claim 19 wherein the screws comprise pores extending therein configured for enabling bone growth to extend into the pores to assist in retaining the screws to the jawbone.
